(19) United States
(12) Patent Application Publication
(10) Pub. No.: US 2023/0147608 A1
(43) Pub. Date: May 11, 2023

(54) HALOGENATED ZINC PHTHALOCYANINE PIGMENT AND PRODUCTION METHOD THEREFOR

(71) Applicant: DIC Corporation, Tokyo (JP)

(72) Inventors: Keisuke Sakamoto, Sakura-shi, Chiba (JP); Ayaka Yamaji, Sakura-shi, Chiba (JP); Kazushi Suzuki, Sakura-shi, Chiba (JP); Mayumi Tokuoka, Kamisu-shi, Ibaraki (JP); Katsunori Shimada, Sakura-shi, Chiba (JP)

(73) Assignee: DIC Corporation, Tokyo (JP)

(21) Appl. No.: 17/296,665

(22) PCT Filed: Apr. 30, 2020
(86) PCT No.: PCT/JP2020/018353

§ 371 Date: May 25, 2021

(51) Int. Cl. C09B 47/10; C09B 67/20; C07D 487/22

(52) U.S. Cl. CPC C09B 47/10; C09B 67/0067; C07D 487/22

(57) ABSTRACT

A method for producing a halogenated zinc phthalocyanine pigment according to one embodiment includes a first step of obtaining a halogenated zinc phthalocyanine crude pigment by deposition by extracting a halogenated zinc phthalocyanine, which is synthesized by using a compound that generates an acid by reacting with water; and a second step of forming the halogenated zinc phthalocyanine crude pigment into a pigment.

### TECHNICAL FIELD

The present invention relates to a halogenated zinc phthalocyanine pigment and a production method therefor.

### BACKGROUND ART

Presently, colored compositions are used in a variety of fields, and examples of the specific usages of the colored compositions include printing inks, paints, coloring agents for resins, coloring agents for fibers, and coloring materials (color filters, toners, and ink jet) for IT information recording. Color matters used in the colored compositions are mainly roughly categorized into pigments and dyes, and organic pigments, which are considered to be superior for their coloring power, have drawn much attention.

Organic compounds that constitute organic pigments undergo aggregation of fine particles after synthesis and are in an aggregated state that is referred to as crude. Thus, usually, synthesized organic compounds cannot be directly used as pigments, and are subjected to a pigmentation step for adjusting the particle size. The aggregate (crude) of an organic compound to be formed into a pigment in the pigmentation step is called a crude pigment, and a fine organic pigment can be obtained by grinding the crude pigment by kneading or the like.

As organic pigments, halogenated zinc phthalocyanine pigments that are used in green pixel portions of color filters etc., have gained much attention (for example, see PTL 1).

### CITATION LIST

### Patent Literature

PTL 1: International Publication No. 2018/043548 pamphlet

### SUMMARY OF INVENTION

### Technical Problem

One of the objects of the present invention is to provide a novel method for producing a halogenated zinc phthalocyanine pigment, with which pigment particles can be made finer.

### Solution to Problem

As a method for synthesizing a halogenated zinc phthalocyanine, for example, a chlorosulfonic acid method, a fusion method, and the like are known. According to these methods, a halogenated zinc phthalocyanine is synthesized by using a compound that generates an acid by reacting with water. A crude pigment, which is an aggregate of halogenated zinc phthalocyanine, is obtained by depositing the synthesized halogenated zinc phthalocyanine in water or an acidic solution. According to such a method, in general, an acid derived from the aforementioned compound that generates an acid by reacting with water adheres to the crude pigment; thus, prior to forming the crude pigment into a pigment, washing for removing the acid adhering to the crude pigment is performed. However, the results of examination by the present inventors have revealed that even when the crude pigment is washed to an extent that the pH of the filtrate is substantially equal to the pH of the water used in washing, the acid still remains inside the crude pigment. The present invention has been made on the basis of such examination results.

That is, an aspect of the present invention relates to a method for producing a halogenated zinc phthalocyanine pigment, the method including a step of obtaining a halogenated zinc phthalocyanine crude pigment by deposition by extracting a halogenated zinc phthalocyanine, which is synthesized by using a compound that generates an acid by reacting with water, in a basic aqueous solution; and a step of forming the halogenated zinc phthalocyanine crude pigment into a pigment.

According to the production method of the aforementioned aspect, encapsulation of acids in the halogenated zinc phthalocyanine crude pigment can be suppressed, and thus a fine halogenated zinc phthalocyanine pigment can be obtained. Thus, according to the production method of the aforementioned aspect, a halogenated zinc phthalocyanine pigment having a large base adsorption amount can be obtained.

In one embodiment, the concentration of a basic compound contained in the basic aqueous solution may be 1 mass % or more.

In one embodiment, the basic aqueous solution may contain a hydroxide of an alkali metal or alkaline earth metal.

In one embodiment, the basic aqueous solution may have a temperature of 5 to 90° C.

In one embodiment, the halogenated zinc phthalocyanine crude pigment may have a pH of 5.0 or more.

In one embodiment, the halogenated zinc phthalocyanine crude pigment may have an Al content of 3000 mass ppm or less.

Another aspect of the present invention relates to a halogenated zinc phthalocyanine pigment having a base adsorption amount of 0.13 mol/kg or more and an Al content of 3000 mass ppm or less.

According to the halogenated zinc phthalocyanine pigment of the aforementioned aspect, the amount of the dispersant used in combination with the pigment can be decreased, and undesirable phenomena caused by blending large quantities of dispersants can be suppressed.

### Advantageous Effects of Invention

According to the present invention, a novel method for producing a halogenated zinc phthalocyanine, with which further micronization of pigment particles is possible, can be provided. In addition, according to the present invention, a halogenated zinc phthalocyanine pigment having a large base adsorption amount and a lower Al content can be obtained.

### DESCRIPTION OF EMBODIMENTS

Preferable embodiments of the present invention will now be described. However, the present invention is not in any way limited by the embodiments described below.

A method for producing a halogenated zinc phthalocyanine pigment according to one embodiment includes a first step of obtaining a halogenated zinc phthalocyanine crude pigment by deposition by extracting a halogenated zinc phthalocyanine, which is synthesized by using a compound that generates an acid by reacting with water, in a basic aqueous solution; and a second step of forming the halogenated zinc phthalocyanine crude pigment into a pigment. Here, the halogenated zinc phthalocyanine is a compound having a structure represented by formula (1) below.

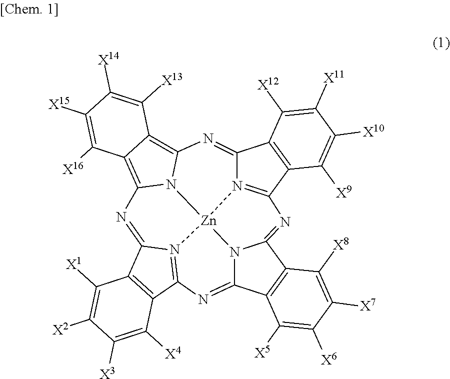

[In formula (1), X¹ to X¹⁶ each independently represent a hydrogen atom or a halogen atom.]

Examples of the halogen atom include a fluorine atom, a chlorine atom, a bromine atom, and an iodine atom. The halogenated zinc phthalocyanine preferably contains, as the halogen atom, at least one of a bromine atom and a chlorine atom, and preferably contains a bromine atom. The halogenated zinc phthalocyanine may contain, as the halogen atom, just one or both of a bromine atom and a chlorine atom. In other words, X¹ to X¹⁶ in formula (1) above may each represent a chlorine atom or a bromine atom.

The first step includes, for example, a synthesis step of synthesizing a halogenated zinc phthalocyanine by using a compound that generates an acid by reacting with water, and a deposition step of performing deposition by extracting the synthesized halogenated zinc phthalocyanine in a basic aqueous solution.

Examples of the method for synthesizing a halogenated zinc phthalocyanine by using a compound that generates an acid by reacting with water include a chlorosulfonic acid method and a fusion method.

An example of the chlorosulfonic acid method is a method that involves dissolving zinc phthalocyanine in a sulfur oxide solvent, such as chlorosulfonic acid, and charging chlorine gas and bromine into the resulting product to perform halogenation. The reaction here is performed at a temperature in the range of 20 to 120° C. for 3 to 20 hours, for example. In the chlorosulfonic acid method, the sulfur oxide solvent, such as chlorosulfonic acid, is the compound that generates an acid by reacting with water. For example, chlorosulfonic acid generates hydrochloric acid and sulfuric acid by reacting with water.

An example of the fusion method is a method that involves halogenating zinc phthalocyanine with a halogenating agent in a melt having a temperature of about 10 to 170° C. and being formed of one compound or two or more compounds that serve as a solvent during halogenation, such as aluminum halide, e.g., aluminum chloride or aluminum bromide, titanium halide, e.g., titanium tetrachloride, alkali metal halide or alkaline earth metal halide such as sodium chloride or sodium bromide (hereinafter referred to as “alkali (earth) metal halide”), or thionyl chloride. In the fusion method, the compound that serves as a solvent during halogenation, such as aluminum halide, titanium halide, alkali (earth) metal halide, or thionyl chloride, is the compound that generates an acid by reacting with water. For example, aluminum chloride generates hydrochloric acid by reacting with water.

A preferable aluminum halide is aluminum chloride. The amount of the aluminum halide added in the aforementioned method that involves using aluminum halide is usually at least three times more and is preferably ten to twenty times more than the amount of zinc phthalocyanine in terms of mole.

An aluminum halide may be used alone; however, combined use of an alkali (earth) metal halide and aluminum halide can further decrease the fusion temperature, and thus provides an operational advantage. A preferable alkali (earth) metal halide is sodium chloride. The amount of the alkali (earth) metal halide added is preferably 1 to 15 parts by mass relative to 10 parts by mass of aluminum halide within the range in which molten salts are generated.

Examples of the halogenating agent include chlorine gas, sulfuryl chloride, and bromine.

The halogenation temperature is preferably 10 to 170° C. and more preferably 30 to 140° C. Furthermore, pressure can be applied to accelerate the reaction. The reaction time may be 5 to 100 hours and is preferably 30 to 45 hours.

The fusion method that uses a combination of two or more of the compounds mentioned above is preferable since the content of a halogenated zinc phthalocyanine having a particular halogen atom composition in the synthesized halogenated zinc phthalocyanine can be freely controlled by adjusting the ratio of the chloride, bromide, and iodide in the molten salt or by changing the amount of introducing chlorine gas, bromine, iodine, or the like and the reaction time. Furthermore, according to the fusion method, decomposition of the raw materials during the reaction is less, the yield from the raw materials is superior, and the reaction can be performed in an inexpensive apparatus without using a strong acid.

In this embodiment, the method for charging raw materials, the type of catalyst and the amount thereof used, the reaction temperature, and the reaction time are optimized, and thus a halogenated zinc phthalocyanine having a halogen atom composition different from existing halogenated zinc phthalocyanines can be obtained.

In the deposition step, for example, a mixture obtained upon completion of the reaction, that is, a mixture containing a halogenated zinc phthalocyanine and a compound that generates an acid by reacting with water, is injected into a basic aqueous solution that serves as an extracting solution so as to settle (deposit) the halogenated zinc phthalocyanine.

According to typical methods, in the deposition step, water or an acidic aqueous solution such as hydrochloric acid is used, not a basic aqueous solution; thus, the acid derived from the compound that generates an acid by reacting with water is incorporated in the deposits. Even when the deposits are washed until, for example, the pH of the filtrate is substantially equal to the pH of water used in washing, the acid (the acid derived from the compound that generates an acid by reacting with water, etc.) incorporated in the deposits is rarely removed, and the acid remains in the crude pigment. The cause for this is presumably as follows: that is, in a halogenated zinc phthalocyanine, the distance between the center metal, zinc, and the nitrogen atoms on the isoindoline units is large, and thus a large void is present around the center metal (zinc); thus, after nitrogen of the phthalocyanine ring is protonated under acidic conditions, the counter anions (for example, chloride ions) smoothly approach the center metal (zinc), and the counter anions and the center metal (zinc) bond to assume a stable structure. Meanwhile, in this embodiment, in the deposition step, a basic aqueous solution is used and thus generation of acids is suppressed or generated acids are neutralized. Thus, it becomes possible to suppress incorporation of the acid in the halogenated zinc phthalocyanine crude pigment.

The mixture containing a halogenated zinc phthalocyanine and a compound that generates an acid by reacting with water contains, for example, 20 to 60 mass % of the halogenated zinc phthalocyanine and 40 to 80 mass % of the compound that generates an acid by reacting with water.

The basic aqueous solution is an aqueous solution having basicity (alkalinity), and is obtained by, for example, dissolving a basic compound in water. Thus, the basic aqueous solution can be rephrased as an aqueous solution containing a basic compound.

The basic compound may be any compound that exhibits basicity in an aqueous solution, and examples thereof include hydroxides of alkali metals or alkaline earth metals such as sodium hydroxide, potassium hydroxide, and calcium hydroxide, carbonates of alkali metals or alkaline earth metals such as sodium carbonate and potassium carbonate, hydrogen carbonates of alkali metals or alkaline earth metals such as sodium hydrogen carbonate, potassium hydrogen carbonate, and calcium hydrogen carbonate, acetates of alkali metals or alkaline earth metals such as sodium acetate, potassium acetate, and calcium acetate, and ammonia. For example, in the fusion method, among these compounds, those which have pKb or 5 or less are preferable and those which have a pKb of 1 or less are more preferable from the viewpoint facilitating removal of aluminum-containing components, such as aluminum hydroxide, that can serve as an inorganic flocculant and further suppressing aggregation of the pigment, and from the viewpoint of further suppressing generation of acids. Specifically, at least one compound selected from the group consisting of hydroxides of alkali metals or alkaline earth metals and acetates of alkali metals or alkaline earth metals is preferably used, a hydroxide of an alkali metal or an alkaline earth metal is more preferably used, and sodium hydroxide is yet more preferably used. Basic compounds can be used alone or in combination.

The concentration of the basic compound contained in the basic aqueous solution is preferably 1 mass % or more, more preferably 3 mass % or more, and yet more preferably 5 mass % or more with respect to the total mass of the basic aqueous solution from the viewpoint of further suppressing generation of acids. The concentration of the basic compound contained in the basic aqueous solution is preferably 30 mass % or less, more preferably 20 mass % or less, and yet more preferably 15 mass % or less with respect to the total mass of the basic aqueous solution from the viewpoint of preventing coarsening of particles.

The amount of the basic compound contained in the basic aqueous solution is preferably 100 parts by mass or more, more preferably 200 parts by mass or more, and yet more preferably 300 parts by mass or more relative to 100 parts by mass of the compound that generates an acid by reacting with water and is contained in the mixture to be injected into the basic aqueous solution from the viewpoint of more sufficiently suppressing generation of acids. The amount of the basic compound contained in the basic aqueous solution is preferably 600 parts by mass or less, more preferably 500 parts by mass or less, and yet more preferably 400 parts by mass or less relative to 100 parts by mass of the compound that generates an acid by reacting with water and is contained in the mixture to be injected into the basic aqueous solution from the viewpoint of preventing coarsening of the particles.

The pH of the basic aqueous solution at 25° C. is preferably 8 or more, more preferably 10 or more, and yet more preferably 13 or more from the viewpoint of further suppressing generation of acids. The pH of the basic aqueous solution 25° C. may be 14 or less.

The temperature of the basic aqueous solution is preferably 1° C. or more, more preferably 5° C. or more, and yet more preferably 10° C. or more from the viewpoint of further suppressing generation of acids. The temperature of the basic aqueous solution is preferably 90° C. or less, more preferably 60° C. or less, and yet more preferably 30° C. or less from the viewpoint of preventing coarsening of particles.

The amount of the basic aqueous solution used is preferably 500 parts by mass or more, more preferably 800 parts by mass or more, and yet more preferably 1000 parts by mass or more relative to 100 parts by mass of the amount of the mixture containing the halogenated zinc phthalocyanine and the compound that generates an acid by reacting with water from the viewpoint of sufficiently depositing the halogenated zinc phthalocyanine. The amount of the basic aqueous solution used is preferably 5000 parts by mass or less, more preferably 3000 parts by mass or less, and yet more preferably 2000 parts by mass or less relative to 100 parts by mass of the mixture containing the halogenated zinc phthalocyanine and the compound that generates an acid by reacting with water from the viewpoint of deflocculating the aggregated particles at a high shear force.

The first step preferably further includes a posttreatment step of post-treating the deposits after the deposition step.

The first step may, for example, further include a step of filtering the deposits (first posttreatment step). The first posttreatment step may be a step that involves filtering and washing the deposits or a step of filtering, washing, and drying the deposits. Washing may be performed by using, for example, an aqueous solvent such as water, sodium hydrogen sulfate water, sodium hydrogen carbonate water, or sodium hydroxide water. In washing, an organic solvent such as acetone, toluene, methyl alcohol, ethyl alcohol, or dimethylformamide may be used as needed. For example, after washing with an aqueous solvent, washing may be performed by using an organic solvent. Washing may be repeated multiple times (for example, two to five times). Specifically, washing is preferably performed until the pH of the filtrate becomes about the same (for example, the difference of 0.2 or less) as the pH of water used for washing.

The first step may, for example, further include a step of dry-grinding the deposits (second posttreatment step). Dry-grinding may be performed in a crusher, for example, an attritor, a ball mill, a vibrating mill, or a vibrating ball mill. Dry crushing may be performed while heating (for example, while heating so that the temperature inside the crusher is 40° C. to 200° C.). After dry-grinding, washing with water may be performed. When washing with water is performed after dry grinding (in particular, after dry grinding by using an attritor), the amount of acids encapsulated in the crude pigment can be further decreased. Washing may involve washing with cold water (washing with water having a temperature of less than 40° C.) or washing with hot water (washing with water having a temperature of 40° C. or more). As in the first posttreatment step, washing is preferably performed until the pH of the filtrate is substantially equal to (for example, a difference of 0.2 or less) the pH of water used for washing. During or prior to washing with water, a treatment for improving wettability of the deposits (for example, a treatment involving causing the deposits to contact a water-soluble organic solvent such as methanol) may be performed. Dry grinding and washing may be performed repeatedly multiple times.

The first step may, for example, further include a step of kneading the deposits together with water (third posttreatment step). The third posttreatment step can further decrease the amount of acids encapsulated in the crude pigment. Kneading can involve the use of a kneader, a mix muller, or the like. Kneading may be performed while heating. For example, the temperature of water may be 40° C. or more. An inorganic salt may be added to water. Here, the force applied during kneading can be improved by allowing at least a portion of the inorganic salt to exist in a solid form. Although an organic solvent (for example, an organic solvent that can be used in the second step described below) can be used during kneading, the amount of the organic solvent used is preferably smaller than the amount of water used, and more preferably, the organic solvent is not used. After kneading, washing may be performed as in the first posttreatment step. Kneading and washing may be performed repeatedly multiple times.

The first step may, for example, further include a step of heating (for example, boiling) the deposits in water (fourth posttreatment step). The fourth posttreatment step can further decrease the amount of acids encapsulated in the crude pigment. The heating temperature in water may be, for example, 40° C. or more and the boiling point or less, and the heating time may be, for example, 1 to 300 minutes. An organic solvent (for example, an organic solvent that can be used in the second step described below) may be mixed in the water; however, the amount of the organic solvent mixed relative to 100 parts by mass of water is preferably 20 parts by mass or less. In the fourth posttreatment step, from the viewpoint of further removing the acids, washing may be performed after the deposits are heated in water, or, after the deposits are heated in water and then washed, heating in water and washing may be performed again at least once (preferably twice or more) repeatedly. Washing may be performed in the same manner as in the first posttreatment step.

In this embodiment, two or more steps selected from the aforementioned first to fourth posttreatment steps may be performed. When two or more steps selected from the first to fourth posttreatment steps are to be performed, the order thereof is not particularly limited.

The halogenated zinc phthalocyanine crude pigment is obtained through the first step; however, as described above, in this embodiment, the deposits obtained in the first step may be directly used as the halogenated zinc phthalocyanine crude pigment, or the deposits may be subjected to the aforementioned posttreatment step (at least one step selected from the first to fourth posttreatment steps) and used as the halogenated zinc phthalocyanine crude pigment.

The halogenated zinc phthalocyanine crude pigment obtained in the first step contains one halogenated zinc phthalocyanine or multiple halogenated zinc phthalocyanines among which the number of halogen atoms is different.

In one embodiment, the average number of bromine atoms in one molecule of the compound represented by formula (1) in the halogenated zinc phthalocyanine crude pigment is less than 13. The average number of bromine atoms may be 12 or less, or 11 or less. The average number of bromine atoms may be 0.1 or more, 6 or more, or 8 or more. The upper limits and the lower limits described above can be freely combined. For example, the average number of bromine atoms may be 0.1 or more but less than 13, may be 8 to 12, or may be 8 to 11. In the description below also, the upper limits and the lower limits described separately may be freely combined.

When the average number of bromine atoms is less than 13, the average number of halogen atoms in one molecule of the compound represented by formula (1) in the halogenated zinc phthalocyanine crude pigment may be 14 or less, 13 or less, less than 13, or 12 or less. The average number of halogen atoms may be 0.1 or more, 8 or more, or 10 or more.

When the average number of bromine atoms is less than 13, the average number of chlorine atoms in one molecule of the compound represented by formula (1) in the halogenated zinc phthalocyanine crude pigment may be 5 or less, 3 or less, 2.5 or less, or less than 2. The average number of chlorine atoms may be 0.1 or more, 0.3 or more, 0.6 or more, 0.8 or more, 1 or more, 1.3 or more, or 2 or more.

In another embodiment, the average number of bromine atoms in one molecule of the compound represented by formula (1) in the halogenated zinc phthalocyanine crude pigment is 13 or more. The average number of bromine atoms may be 14 or more. The average number of bromine atoms may be 15 or less.

When the average number of bromine atoms is 13 or more, the average number of halogen atoms in one molecule of the compound represented by formula (1) in the halogenated zinc phthalocyanine crude pigment may be 13 or more, 14 or more, or 15 or more. The average number of halogen atoms may be 16 or less or 15 or less.

When the average number of bromine atoms is 13 or more, the average number of chlorine atoms in one molecule of the compound represented by formula (1) in the halogenated zinc phthalocyanine crude pigment may be 0.1 or more or 1 or more. The average number of chlorine atoms may be 3 or less, or less than 2.

The number of halogen atoms mentioned above (for example, the number of bromine atoms and the number of chlorine atoms) can be determined, for example, by mass spectrometry of a halogenated zinc phthalocyanine crude pigment by using a matrix-assisted laser desorption/ionization time-of-flight mass spectrometer (JMS-S3000 produced by JEOL Ltd., or the like). Specifically, the numbers of respective halogen atoms can be calculated as relative values per zinc atom from the mass ratio between zinc atoms to the halogen atoms in the halogenated zinc phthalocyanine crude pigment.

The arithmetic standard deviation of the particle size distribution of the halogenated zinc phthalocyanine crude pigment is, for example, 15 nm or more. The arithmetic standard deviation of the particle size distribution of the halogenated zinc phthalocyanine crude pigment is, for example, 1500 nm or less. When the arithmetic standard deviation of the particle size distribution of the halogenated zinc phthalocyanine crude pigment is within such a range, finer pigment particles are easily obtained. The arithmetic standard deviation of the particle size distribution of the halogenated zinc phthalocyanine crude pigment can be measured by using a dynamic light scattering particle diameter distribution measurement instrument, and, specifically, can be measured by the following method under the following conditions.

### <Method>

In a paint shaker produced by TOYO SEIKI CO., LTD., 2.48 g of a halogenated zinc phthalocyanine crude pigment, 1.24 g of BYK-LPN6919 produced by BYK-Chemie, 1.86 g of UNIDIC ZL-295 produced by DIC Corporation, and 10.92 g of propylene glycol monomethyl ether acetate are dispersed with zircon beads having a diameter of 0.3 to 0.4 mm for 2 hours to obtain a dispersion. After the zircon beads are removed by using a nylon mesh, 0.02 g of the obtained dispersion is diluted with 20 g of propylene glycol monomethyl ether acetate to obtain a particle size distribution measurement dispersion.

### <Conditions>

Measurement instrument: dynamic light scattering particle diameter distribution analyzer LB-550 (produced by Horiba Ltd.)

Measurement temperature: 25° C.

Measurement sample: particle size distribution measurement dispersion

Data analysis conditions: particle diameter-based scattering light intensity, dispersion medium refractive index: 1.402

The halogenated zinc phthalocyanine crude pigment obtained in this embodiment contains less encapsulated acids compared to typical crude pigments. Thus, the pH of the halogenated zinc phthalocyanine crude pigment is, for example, 4.0 or more. Here, the pH of the halogenated zinc phthalocyanine crude pigment can be confirmed by mixing 5 g of the halogenated zinc phthalocyanine crude pigment with 5 g of methanol, mixing the resulting mixture with 100 ml of ion exchange water, heating the resulting mixture for 5 minutes to boil, maintaining the boiling state for 5 minutes by further heating, cooling the heated mixture to 30° C. or lower, adjusting the total amount of the mixture to 100 ml by using ion exchange water, filtering the resulting mixture, and measuring the pH of the resulting filtrate at 25° C. From the viewpoint of more easily obtaining finer pigment particles, the pH of the halogenated zinc phthalocyanine crude pigment is preferably 5.0 or more, more preferably 5.5 or more, yet more preferably 6.0 or more, and still more preferably 6.5 or more. The pH of the halogenated zinc phthalocyanine crude pigment may be, for example, 8.5 or less, 8.0 or less, or 7.5 or less.

The halogenated zinc phthalocyanine crude pigment sometimes encapsulates aluminum-containing components such as aluminum hydroxide when a compound containing an aluminum halide is used in the fusion method, for example. However, the aluminum-containing components can cause contrast degradation, and thus the aluminum content (Al content) in the halogenated zinc phthalocyanine crude pigment is preferably as small as possible. From this viewpoint, the Al content contained in the halogenated zinc phthalocyanine crude pigment is preferably 3000 mass ppm or less, preferably 2000 mass ppm or less, and yet more preferably 1000 mass ppm or less. The Al content in the halogenated zinc phthalocyanine crude pigment can be determined by a high-frequency inductively coupled plasma optical emission spectrometry (ICP optical emission spectrometry). When a strongly basic aqueous solution is used in the deposition step in the first step, aluminum hydroxide can be dissolved and removed, and thus the Al content tends to be decreased.

In the second step, for example, the halogenated zinc phthalocyanine crude pigment is kneaded and ground to achieve micronization. Kneading may involve the use of a kneader, a mix muller, or the like.

The second step may be a step of kneading the halogenated zinc phthalocyanine crude pigment together with an organic solvent, or may be a step of kneading the halogenated zinc phthalocyanine crude pigment together with an inorganic salt and an organic solvent. In the second step, water is preferably not used. The amount of water used may be, for example, 20 parts by mass or less, 10 parts by mass or less, or 5 parts by mass or less relative to 100 parts by mass of the halogenated zinc phthalocyanine crude pigment.

An organic solvent that does not dissolve the halogenated zinc phthalocyanine crude pigment and the inorganic salt can be used as the organic solvent. An organic solvent that can suppress crystal growth is preferably used as the organic solvent. A water-soluble organic solvent is suitable for use as such an organic solvent. Examples of the organic solvent that can be used include diethylene glycol, glycerin, ethylene glycol, propylene glycol, liquid polyethylene glycol, liquid polypropylene glycol, 2-(methoxymethoxy)ethanol, 2-butoxyethanol, 2-(isopentyloxy) ethanol, 2-(hexyloxy)ethanol, diethylene glycol monomethyl ether, diethylene glycol monoethyl ether, diethylene glycol monobutyl ether, triethylene glycol, triethylene glycol monomethyl ether, 1-methoxy-2-propanol, 1-ethoxy-2-propanol, dipropylene glycol, dipropylene glycol monomethyl ether, and dipropylene glycol monoethyl ether. The amount of the organic solvent (for example, a water-soluble organic solvent) used is not particularly limited but is preferably 1 to 500 parts by mass relative to 100 parts by mass of the halogenated zinc phthalocyanine crude pigment.

In the second step, the halogenated zinc phthalocyanine crude pigment may be kneaded while being heated. From the viewpoint of easily obtaining finer pigment particles, the heating temperature is preferably 40° C. or more, more preferably 60° C. or more, yet more preferably 80° C. or more, and still more preferably 90° C. or more. The heating temperature may be, for example, 150° C. or less.

The kneading time in the second step may be, for example, 1 to 60 hours.

In the second step, when an inorganic salt and an organic solvent are used, a mixture containing the halogenated zinc phthalocyanine pigment, the inorganic salt, and the organic solvent is obtained; however, the organic solvent and the inorganic salt may be removed from this mixture, and a solid matter mainly composed of the halogenated zinc phthalocyanine pigment may be subjected to operations such as washing, filtering, drying, crushing, and the like as needed.

In washing, either cold water or hot water may be used. Washing may be repeated once to five times. When a water-soluble inorganic salt and a water-soluble organic solvent are used, the organic solvent and the inorganic salt can be easily removed by washing with water. If needed, acid washing, alkali washing, or organic solvent washing may be performed.

Examples of drying after washing and filtering include batch-drying and continuous drying that involve removing water and/or solvent of the pigment by performing heating at 80 to 120° C. using a heating source installed in a dryer. Examples of the dryer include a box dryer, a band dryer, and a spray dryer. In particular, spray drying using a spray dryer is preferable since dispersing is easy during paste preparation.

Crushing after drying is performed not to increase the specific surface area or decrease the average particle diameter of primary particles but is performed to pulverize and disintegrate the lump-like pigment obtained when, for example, a box dryer or a band dryer is used for drying. Examples of crushing include crushing by using a mortar, a hammer mill, a disk mill, a pin mill, or a jet mill.

According to the aforementioned production method, a fine halogenated zinc phthalocyanine pigment can be obtained. The present inventors presume the reason why such an effect is obtained as follows. First, when acids are present during pigmentation, acids accelerate aggregation of particles and thus micronization of pigment particles is inhibited. Meanwhile, in the production method described above, encapsulation of acids in the crude pigment is suppressed, and thus the influence of acids can be alleviated. Thus, according to the aforementioned method, a fine halogenated zinc phthalocyanine pigment is obtained.

The halogenated zinc phthalocyanine pigment obtained by the aforementioned production method is suitable for use as a green pigment for color filters. In general, contrast and brightness tend to improve as the particles of the pigment used in pixel portions of a color filter become smaller. Thus, when the halogenated zinc phthalocyanine pigment obtained by the aforementioned production method is used as a green pigment for color filters, excellent contrast tends to be obtained, and excellent brightness tends to be obtained.

The average particle diameter of the primary particles (average primary particle diameter) of the halogenated zinc phthalocyanine pigment obtained by the aforementioned method is, for example, 30 nm or less. According to the aforementioned method, a halogenated zinc phthalocyanine pigment that has an average primary particle diameter of 25 nm or less, for example, can be obtained. The average primary particle diameter of the halogenated zinc phthalocyanine pigment may be 10 nm or more. Here, the average primary particle diameter is an average value of long axes of primary particles, and can be determined by measuring the long axes of the primary particles in a manner similar to measuring the average aspect ratio described below.

The average aspect ratio of the primary particles of the halogenated zinc phthalocyanine pigment is, for example, 1.2 or more, 1.3 or more, 1.4 or more, or 1.5 or more. The average aspect ratio of the primary particles of the halogenated zinc phthalocyanine pigment is, for example, less than 2.0, 1.8 or less, 1.6 or less, or 1.4 or less. Better contrast is obtained by using a halogenated zinc phthalocyanine pigment having such an average aspect ratio.

The halogenated zinc phthalocyanine pigment that has a primary particle average aspect ratio within the range of 1.0 to 3.0 preferably does not contain primary particles having an aspect ratio of 5 or more, more preferably does not contain primary particles having an aspect ratio of 4 or more, and further more preferably does not contain primary particles having an aspect ratio exceeding 3.

The aspect ratios and the average aspect ratio of the primary particles can be measured by the following method. First, particles in a view area are imaged with a transmission electron microscope (for example, JEM-2010 produced by JEOL Ltd.). For each of the primary particles present in a two-dimensional image, an axis that is long (long axis) and an axis that is short (short axis) are measured, and the ratio of the long axis to the short axis is assumed to be the aspect ratio of that primary particle. The average values of the long axes and the short axes are determined for forty primary particles, and the ratio of the long axis to the short axis is calculated using the determined values and is assumed to be the average aspect ratio. In this process, the halogenated zinc phthalocyanine pigment used as a sample is ultrasonically dispersed in the solvent (for example, cyclohexane) and then imaged with a microscope. Alternatively, a scanning electron microscope may be used instead of a transmission electron microscope.

Since the production method described above can suppress aggregation of the pigment and reduce the size of the primary particles of the pigment, the surface area to which bases can adsorb can be increased. Thus, according to the aforementioned production method, a halogenated zinc phthalocyanine pigment having a large base adsorption amount can be obtained. When a halogenated zinc phthalocyanine pigment is dispersed, a dispersant having basic functional groups (for example, primary to tertiary amino groups) as pigment-adsorbing groups is widely used; however, when the halogenated zinc phthalocyanine pigment has a large base adsorption amount, the amount of such a dispersant used can be decreased. Thus, the halogenated zinc phthalocyanine pigment having a large base adsorption amount can suppress undesirable phenomena such as degradation of the contrast and brightness caused by decomposition of a dispersant, which has lower heat resistance the pigment, due to the heat at around 200° C. during production of a color filter, degradation of the resolution and developing property caused by the dispersant insoluble in the developing solution, and an increase in the thickness of the color filter caused by the dispersant that serves as a non-coloring component.

Thus, according to the aforementioned production method, the Al content in the halogenated zinc phthalocyanine crude pigment can be decreased by using a strongly basic aqueous solution in the deposition step in a fusion method or the like. Usually, the Al content rarely changes during pigmentation, and thus there is a tendency that a halogenated zinc phthalocyanine pigment having a small Al content and a large base adsorption amount is obtained from such a halogenated zinc phthalocyanine crude pigment having a small Al content. For example, a halogenated zinc phthalocyanine pigment having a base adsorption amount of 0.13 mol/kg or more and an Al content of 3000 mass ppm or less can be obtained.

The base adsorption amount of the halogenated zinc phthalocyanine pigment is preferably 0.13 mol/kg or more, more preferably 0.135 mol/kg or more, and still more preferably 0.140 mol/kg or more. The base adsorption amount of the halogenated zinc phthalocyanine pigment may be 0.160 mol/kg or less. The base adsorption amount is measured by the method described in Examples.

The Al content in the halogenated zinc phthalocyanine pigment is preferably 3000 mass ppm or less, more preferably 2000 mass ppm or less, still more preferably 1000 mass ppm or less, and particularly preferably less than 1000 mass ppm. The Al content in the halogenated zinc phthalocyanine pigment can be determined by a high-frequency inductively coupled plasma optical emission spectrometry (ICP optical emission spectrometry).

### EXAMPLES

The contents of the present invention will now be described in further details through examples and comparative examples that do not limit the present invention.

### Example 1

[Synthesis of Crude Pigment]

Into a 300 ml flask, 91 g of sulfuryl chloride (produced by FUJIFILM Wako Pure Chemical Corporation), 109 g of aluminum chloride (produced by KANTO CHEMICAL CO., INC.), 15 g of sodium chloride (produced by Tokyo Chemical Industry Co., Ltd.), 30 g of zinc phthalocyanine (produced by DIC Corporation), and 230 g of bromine (produced by FUJIFILM Wako Pure Chemical Corporation) were charged. The temperature was elevated to 130° C. and retained at 130° C. for 40 hours. After the reaction mixture was extracted into 2500 g of an aqueous sodium oxide (NaOH) solution having a liquid temperature of 15° C. and a concentration of 10 mass %, the reaction mixture was filtered, washed with water, and dried to obtain a halogenated zinc phthalocyanine crude pigment (crude pigment A1). Washing with water was performed until the difference between the pH of the filtrate and the pH of the water used in washing was ±0.2.

The crude pigment A1 was subjected to mass spectrometry with JMS-S3000 produced by JEOL Ltd., and it was confirmed that the halogenated zinc phthalocyanine (P1) constituting the crude pigment A1 had an average of 1.8 chlorine atoms and an average of 13.2 bromine atoms. The delay time for the mass spectrometry was 500 ns, the laser intensity was 44%, and the resolving power value of the peak in m/z=1820 or more and 1860 or less was 32111.

[Measurement of pH of Crude Pigment A1]

Into a 300 ml beaker, 5 g of the crude pigment A1 and 5 g of methanol were weighed and placed, and the resulting mixture was mixed. Thereto, 100 ml of ion exchange water was weighed and added, the resulting mixture was brought to boil over a period of 5 minutes using a hot stirrer, and boiling was continued further for 5 minutes. Next, after cooled to 30° C. or less, the mixture was placed in a 100 ml measuring cylinder, the total amount was adjusted to 100 ml by using ion exchange water, and the resulting mixture was filtered. The pH and the specific conductivity of the filtrate were measured, and it was found that the pH of the crude pigment A1 at 25° C. was 7.8, and the specific conductivity was 67 μS/cm (microsiemens per centimeter). The pH was measured with a personal pH meter PH71 produced by Yokogawa Electric Corporation, and the specific conductivity was measured with SevenEasy S30 produced by METTLER TOLEDO.

[Measurement of Aluminum (Al) Content in Crude Pigment A1]

With 5 ml of nitric acid, 0.25 g of the crude pigment A1 was mixed, the resulting mixture was irradiated with microwaves to be decomposed, and the volume of the resulting product was adjusted to 25 ml by using ion exchange water. To an ICP optical emission spectrometry aluminum standard solution, nitric acid in an amount about the same as that used in pigment decomposition was added, six samples for calibration curve plotting, i.e., samples for 0 mass ppm, 1000 mass ppm, 2000 mass ppm, 5000 mass ppm, 10000 mass ppm, and 100000 mass ppm were prepared, the Al content was measured with an IPC spectrometer (Optima 4300DV produced by Perkin Elmer), and a calibration curve was drawn. The solution composed of the pigment decomposition product was also analyzed with the ICP spectrometer, and the Al content in the crude pigment A1 was calculated from the calibration curve. The Al content was 1000 mass ppm or less.

[Pigmentation]

Into a double-armed kneader, 40 g of the crude pigment A1, 400 g of crushed sodium chloride, and 63 g of diethylene glycol (DEG) were charged, and the resulting mixture was kneaded at 80° C. for 8 hours. The kneaded mixture was extracted into 2 kg of water at 80° C., and stirred for 1 hour. Subsequently, filtering, washing with hot water, drying, and crushing were performed to obtain a green pigment G1.

[Measurement of Average Primary Particle Diameter]

The green pigment G1 was ultrasonically dispersed in cyclohexane and imaged with a microscope, and the average particle diameter of primary particles (average primary particle diameter) was calculated from the average of forty primary particles constituting the aggregates in the two-dimensional image. The average particle diameter of the primary particles was 24 nm.

[Measurement of pH of Green Pigment G1]

Into a 300 ml beaker, 5 g of the green pigment G1 and 5 g of methanol were weighed and placed, and the resulting mixture was mixed. Thereto, 100 ml of ion exchange water was weighed and added, the resulting mixture was brought to boil over a period of 5 minutes using a hot stirrer, and boiling was continued further for 5 minutes. Next, after cooled to 30° C. or less, the mixture was placed in a 100 ml measuring cylinder, the total amount was adjusted to 100 ml by using ion exchange water, and the resulting mixture was filtered. The pH and the specific conductivity of the filtrate were measured and were 7.6 at 25° C. and 59 μS/cm, respectively.

[Measurement of Aluminum (Al) Content in Green Pigment G1]

The Al content in the green pigment G1 was measured in the same manner as measuring the Al content in the crude pigment A1 except that the green pigment G1 was used instead of the crude pigment A1. The Al content was 1000 mass ppm.

[Measurement of Base Adsorption Amount]

The base adsorption amount of the green pigment G1 was measured by using an automatic titrator COM-1700 (produced by Hitachi High-Tech Science Corporation). Specifically, first, about 0.1 g of the green pigment G1 and 15 mL of a base solution for adsorption were mixed and stirred with a conditioning mixer (2000 rpm, 3 minutes). After the green pigment G1 was settled by centrifugal separation (11000 rpm, 20 minutes), 10 mL of the supernatant was taken and diluted with 50 mL of n-propyl acetate (NPAC), and the resulting solution was subjected to potentiometric titration to measure the amount of non-adsorbing bases present in the supernatant solution. The determined amount of the non-adsorbing bases was subtracted from the amount of the bases added so as to calculate the amount of the base adsorbed to the green pigment G1. As the base solution for adsorption, a 0.001 mol/L tetra-n-butylammonium hydroxide (TBAH)/NPAC solution was used, and, as the acidic solution for titration, a 0.001 mol/L p-toluenesulfonic acid (PTSA)/NPAC solution was used.

[Evaluation of Contrast and Brightness]

In a paint shaker produced by TOYO SEIKI CO., LTD., 1.65 g of pigment yellow 138 (CHROMOFINE YELLOW 6206EC produced by Dainichiseika Color & Chemicals Mfg. Co., Ltd.), 3.85 g of DISPERBYK-161 (produced by BYK-Chemie), and 11.00 g of propylene glycol monomethyl ether acetate were dispersed together with zircon beads having a diameter of 0.3 to 0.4 mm for 2 hours to obtain a dispersion.

In a paint shaker, 4.0 g of the aforementioned dispersion, 0.98 g of UNIDIC ZL-295, and 0.22 g of propylene glycol monomethyl ether acetate were added and mixed to obtain a yellow composition (TY1) for toning.

In a paint shaker produced by TOYO SEIKI CO., LTD., 2.48 g the green pigment G1 obtained in Example 1, 1.24 g of BYK-LPN6919 produced by BYK-Chemie, 1.86 g of UNIDIC ZL-295 produced by DIC Corporation, and 10.92 g of propylene glycol monomethyl ether acetate were dispersed together with zircon beads having a diameter of 0.3 to 0.4 mm for 2 hours to obtain a color filter pigment dispersion (MG1).

In a paint shaker, 4.0 g of the color filter pigment dispersion (MG1), 0.98 g of UNIDIC ZL-295 produced by DIC Corporation, and 0.22 g of propylene glycol monomethyl ether acetate were added and mixed to obtain an evaluation composition (CG1) for forming green pixel portions of a color filter.

The evaluation composition (CG1) was applied to a soda glass substrate by spin coating, dried at 90° C. for 3 minutes, and heated at 230° C. for 1 hour. As a result, a glass substrate that has a colored film on a soda glass substrate and is used for evaluating contrast was prepared. The thickness of the colored film obtained by heating at 230° C. for 1 hour was adjusted to 1.8 μm by adjusting the spinning rate during spin coating.

A coating solution obtained by mixing the yellow composition (TY1) for toning and the evaluation composition (CG1) described above was applied to a soda glass substrate by spin coating, dried at 90° C. for 3 minutes, and heated at 230° C. for 1 hour. As a result, a glass substrate that has a colored film on a soda glass substrate and is used for evaluating brightness was prepared. A colored film having a thickness of the colored film having a chromaticity (x, y) of (0.275, 0.570) for a C light source and obtained by heating at 230° C. for 1 hour was prepared by adjusting the mixing ratio of the yellow composition (TY1) for toning and the evaluation composition (CG1) and the spinning rate during spin coating.

The contrast of the colored film on the glass substrate for contrast evaluation was measured with a contrast tester CT-1 produced by TSUBOSAKA ELECTRIC Co., Ltd., and the brightness of the colored film on the glass substrate for brightness evaluation was measured with U-3900 produced by Hitachi High-Tech Corporation. The results are shown in Table 1. Note that the contrast and brightness shown in Table 1 are values based on the contrast and brightness of Comparative Example 1.

### Examples 2 and 3

Crude pigments A2 and A3 were obtained as in Example 1 except that, during synthesis of the crude pigment, an aqueous sodium acetate (CH₃COONa) solution or an aqueous sodium hydrogen carbonate (NaHCO₃) solution was used as the extracting solution instead of the aqueous sodium hydroxide solution. The crude pigments A2 and A3 were subjected to mass spectrometry with JMS-S3000 produced by JEOL Ltd., and were each confirmed to be constituted by a halogenated zinc phthalocyanine (Pl) having an average of 1.8 chlorine atoms and an average of 13.2 bromine atoms. Moreover, the pH and the specific conductivity of the crude pigments A2 and A3 and the Al content in the crude pigments A2 and A3 were measured as in Example 1. The results are shown in Table 1.

Green pigments G2 and G3 were obtained as in Example 1 except that the crude pigment A2 or A3 was used instead of the crude pigment A1. Moreover, the average primary particle diameter, the pH, the specific conductivity, the Al content, and the base adsorption amount of the green pigments G2 and G3 were measured as in Example 1. A glass substrate for contrast evaluation and a glass substrate for brightness evaluation were prepared as in Example 1 except that the green pigment G2 or G3 was used instead of the green pigment G1, and the contrast and brightness were measured. The results are shown in Table 1.

### Examples 4 to 8

Crude pigments A4 and A8 were obtained as in Example 1 except that, during synthesis of the crude pigment, the concentration of the aqueous sodium hydroxide solution serving as the extracting solution was changed to a value indicated in Table 1. The crude pigments A4 to A8 were subjected to mass spectrometry with JMS-S3000 produced by JEOL Ltd., and were each confirmed to be constituted by a halogenated zinc phthalocyanine (P1) having an average of 1.8 chlorine atoms and an average of 13.2 bromine atoms. Moreover, the pH and the specific conductivity of the crude pigments A4 to A8 and the Al content in the crude pigments A4 to A8 were measured as in Example 1. The results are shown in Table 1.

Green pigments G4 to G8 were obtained as in Example 1 except that the crude pigments A4 to A8 were respectively used instead of the crude pigment A1. Moreover, the average primary particle diameter, the pH, the specific conductivity, the Al content, and the base adsorption amount of the green pigments G4 to G8 were measured as in Example 1. A glass substrate for contrast evaluation and a glass substrate for brightness evaluation were prepared as in Example 1 except that the green pigments G4 to G8 were used instead of the green pigment G1, and the contrast and brightness were measured. The results are shown in Table 1.

### Examples 9 and 10

Crude pigments A9 and A10 were obtained as in Example 1 except that, during synthesis of the crude pigment, the temperature of the aqueous sodium hydroxide solution serving as the extracting solution was changed to a value indicated in Table 1. The crude pigments A9 and A10 were subjected to mass spectrometry with JMSS3000 produced by JEOL Ltd., and were each confirmed to be constituted by a halogenated zinc phthalocyanine (P1) having an average of 1.8 chlorine atoms and an average of 13.2 bromine atoms. Moreover, the pH and the specific conductivity of the crude pigments A9 and A10 and the Al content in the crude pigments A9 and A10 were measured as in Example 1. The results are shown in Table 1.

Green pigments G9 and G10 were obtained as in Example 1 except that the crude pigment A9 or A10 was used instead of the crude pigment A1. Moreover, the average primary particle diameter, the pH, the specific conductivity, the Al content, and the base adsorption amount of the green pigments G9 and G10 were measured as in Example 1. A glass substrate for contrast evaluation and a glass substrate for brightness evaluation were prepared as in Example 1 except that the green pigment G9 or G10 was used instead of the green pigment G1, and the contrast and brightness were measured. The results are shown in Table 1.

### Examples 11 and 12

Green pigments G11 and G12 were obtained as in Example 1 except that, in the pigmentation step, the heating time and/or the kneading time during kneading was changed as indicated in Table 1. Moreover, the average primary particle diameter, the pH, the specific conductivity, the Al content, and the base adsorption amount of the green pigments G11 and G12 were measured as in Example 1. A glass substrate for contrast evaluation and a glass substrate for brightness evaluation were prepared as in Example 1 except that the green pigment G11 or G12 was used instead of the green pigment G1, and the contrast and brightness were measured. The results are shown in Table 1.

### Comparative Examples 1 and 2

Crude pigments A13 and A14 were obtained as in Example 1 except that, during synthesis of the crude pigment, water or hydrochloric acid (aqueous HCl solution) having a concentration of 10 mass % was used instead of sodium hydroxide serving as the extracting solution. The crude pigments A13 and A14 were subjected to mass spectrometry with JMS-S3000 produced by JEOL Ltd., and were each confirmed to be constituted by a halogenated zinc phthalocyanine (P1) having an average of 1.8 chlorine atoms and an average of 13.2 bromine atoms. Moreover, the pH and the specific conductivity of the crude pigments A13 and A14 and the Al content in the crude pigments A13 and A14 were measured as in Example 1. The results are shown in Table 1.

Green pigments G13 and G14 were obtained as in Example 1 except that the crude pigment A13 or A14 was used instead of the crude pigment A1. Moreover, the average primary particle diameter, the pH, the specific conductivity, the Al content, and the base adsorption amount of the green pigments G13 and G14 were measured as in Example 1. A glass substrate for contrast evaluation and a glass substrate for brightness evaluation were prepared as in Example 1 except that the green pigment G13 or G14 was used instead of the green pigment G1, and the contrast and brightness were measured. The results are shown in Table 1.

### Example 13

Into a 300 ml flask, 90 g of sulfuryl chloride (produced by FUJIFILM Wako Pure Chemical Corporation), 105 g of aluminum chloride (produced by KANTO CHEMICAL CO., INC.), 14 g of sodium chloride (produced by Tokyo Chemical Industry Co., Ltd.), 27 g of zinc phthalocyanine (produced by DIC Corporation), and 55 g of bromine (produced by FUJIFILM Wako Pure Chemical Corporation) were charged. The temperature was elevated to 130° C. and retained at 130° C. for 40 hours. After the reaction mixture was extracted into 2500 g of an aqueous sodium oxide (NaOH) solution having a liquid temperature of 15° C. and a concentration of 10 mass %, the reaction mixture was filtered, washed with water, and dried to obtain a halogenated zinc phthalocyanine crude pigment (crude pigment A15). Washing with water was performed until the pH of the filtrate was substantially equal to the pH of the water used in washing.

The crude pigment A15 was subjected to mass spectrometry with JMS-S3000 produced by JEOL Ltd., and it was confirmed that the halogenated zinc phthalocyanine (P2) constituting the crude pigment A15 has an average of 2.9 chlorine atoms and an average of 9.3 bromine atoms. The delay time for the mass spectrometry was 510 ns, the laser intensity was 40%, and the resolving power value of the peak in m/z=1820 or more and 1860 or less was 65086. Moreover, the pH and the specific conductivity of the crude pigment A15 and the Al content in the crude pigment A16 were measured as in Example 1. The results are shown in Table 2.

A green pigment G15 was obtained as in Example 1 except that the crude pigment A15 was used instead of the crude pigment A1. Moreover, the average primary particle diameter, the pH, the specific conductivity, the Al content, and the base adsorption amount of the green pigment G15 were measured as in Example 1. A glass substrate for contrast evaluation and a glass substrate for brightness evaluation were prepared as in Example 1 except that pigment yellow 185 (Paliotol Yellow D1155 produced by BASF) was used instead of pigment yellow 138 (CHROMOFINE YELLOW 6206EC produced by Dainichiseika Color & Chemicals Mfg. Co., Ltd.), that the green pigment G15 was used instead of the green pigment G1, and that the chromaticity (x, y) of the colored film was adjusted to (0.230, 0.670), and the contrast and brightness were measured. The results are shown in Table 2.

### Comparative Example 3

A crude pigment A16 was obtained as in Example 13 except that, during synthesis of the crude pigment, water was used instead of sodium hydroxide serving as the extracting solution. The crude pigment A16 was subjected to mass spectrometry with JMS-S3000 produced by JEOL Ltd., and was confirmed to be constituted by a halogenated zinc phthalocyanine (P2) having an average of 2.9 chlorine atoms and an average of 9.3 bromine atoms. Moreover, the pH and the specific conductivity of the crude pigment A16 and the Al content in the crude pigment A16 were measured as in Example 13. The results are shown in Table 2.

A green pigment G16 was obtained as in Example 13 except that the crude pigment A16 was used instead of the crude pigment A15. Moreover, the average primary particle diameter, the pH, the specific conductivity, the Al content, and the base adsorption amount of the green pigment G17 were measured as in Example 13. A glass substrate for contrast evaluation and a glass substrate for brightness evaluation were prepared as in Example 13 except that the green pigment G16 was used instead of the green pigment G15, and the contrast and brightness were measured. The results are shown in Table 2.

TABLE 1

| | First step | | | | | | | |
|---|---|---|---|---|---|---|---|---|
| | | | | | | Specific | | |
| | | | Extracting | Extracting | | conductivity | Al content | |
| | | | solution | solution | pH of | of crude | of crude | Second step |
| | | Extracting | concentration | temperature | crude | pigment | pigment | Temperature |
| | Compound | solution | (mass %) | (° C.) | pigment | (μS/cm) | (mass ppm) | (° C.) |
| Example 1 | P1 | NaOH aq | 10 | 15 | 7.8 | 87 | <1000 | 80 |
| Example 2 | P1 | CH₃COONa aq | 10 | 15 | 5.4 | 109 | 2000 | 80 |
| Example 3 | P1 | NaHCO₃ aq | 10 | 15 | 4.9 | 131 | >100000 | 80 |
| Example 4 | P1 | NaOH aq | 0.2 | 15 | 4.2 | 111 | <1000 | 80 |
| Example 5 | P1 | NaOH aq | 1 | 15 | 5.0 | 104 | <1000 | 80 |
| Example 6 | P1 | NaOH aq | 2 | 15 | 5.5 | 99 | <1000 | 80 |
| Example 7 | P1 | NaOH aq | 5 | 15 | 6.5 | 86 | <1000 | 80 |
| Example 8 | P1 | NaOH aq | 20 | 15 | 8.4 | 51 | <1000 | 80 |
| Example 9 | P1 | NaOH aq | 10 | 5 | 7.0 | 65 | <1000 | 80 |
| Example 10 | P1 | NaOH aq | 10 | 40 | 7.8 | 61 | <1000 | 80 |
| Example 11 | P1 | NaOH aq | 10 | 15 | 7.8 | 67 | <1000 | 60 |
| Example 12 | P1 | NaOH aq | 10 | 15 | 7.8 | 67 | <1000 | 100 |
| Comparative | P1 | Water | — | 15 | 3.7 | 163 | <1000 | 80 |
| Example 1 | | | | | | | | |
| Comparative | P1 | HCl aq | 10 | 15 | 3.4 | 2280 | <1000 | 80 |
| Example 2 | | | | | | | | |

| | Second step | | | | | | Characteristic values | |
|---|---|---|---|---|---|---|---|---|
| | | | | Primary | Base | | Monochromatic | Y138 toning |
| | | | Specific | particle | adsorption | | evaluation: film | evaluation: |
| | | | conductivity | diameter | amount | Al content | thickness = 1.8 μm | (0.275, 0.579) |
| | Time | pH of | of pigment | of pigment | of pigment | of pigment | Contrast | Brightness |
| | (h) | pigment | (μS/cm) | (nm) | (mol/kg) | (mass ppm) | Std. ratio | Std. ratio |
| Example 1 | 8 | 7.6 | 59 | 24 | 0.143 | <1000 | 132% | 105.3% |
| Example 2 | 8 | 5.5 | 84 | 29 | 0.138 | 2000 | 109% | 104.6% |
| Example 3 | 8 | 4.9 | 102 | 32 | 0.134 | >100000 | 92% | 100.6% |
| Example 4 | 8 | 4.3 | 80 | 32 | 0.138 | <1000 | 103% | 100.2% |
| Example 5 | 8 | 5.1 | 71 | 29 | 0.140 | <1000 | 112% | 104.8% |
| Example 6 | 8 | 5.6 | 74 | 28 | 0.140 | <1000 | 118% | 105.0% |
| Example 7 | 8 | 6.6 | 68 | 26 | 0.143 | <1000 | 123% | 105.0% |
| Example 8 | 8 | 7.7 | 43 | 26 | 0.149 | <1000 | 126% | 105.1% |
| Example 9 | 8 | 7.5 | 52 | 24 | 0.153 | <1000 | 134% | 105.3% |
| Example 10 | 8 | 7.6 | 48 | 25 | 0.142 | <1000 | 125% | 105.2% |
| Example 11 | 15 | 7.7 | 54 | 25 | 0.147 | <1000 | 129% | 105.2% |
| Example 12 | 8 | 7.7 | 52 | 23 | 0.145 | <1000 | 127% | 105.4% |
| Comparative | 8 | 3.7 | 134 | 34 | 0.122 | <1000 | Std. | Std. |
| Example 1 | | | | | | | | |
| Comparative | 8 | 3.5 | 1640 | 35 | 0.109 | <1009 | 86% | 100.3% |
| Example 2 | | | | | | | | |

TABLE 2

| | First step | | | | | | | |
|---|---|---|---|---|---|---|---|---|
| | | | | | | Specific | | |
| | | | Extracting | Extracting | | conductivity | Al content | |
| | | | solution | solution | pH of | of crude | of crude | Second step |
| | | Extracting | concentration | temperature | crude | pigment | pigment | Temperature |
| | Compound | solution | (mass %) | (° C.) | pigment | (μS/cm) | (mass ppm) | (° C.) |
| Example 13 | P2 | NaOH aq | 10 | 15 | 7.9 | 88 | <1000 | 80 |
| Comparative | P2 | Water | — | 15 | 3.4 | 193 | <1000 | 80 |
| Example 3 | | | | | | | | |

| | Second step | | | | | | Characteristic values | |
|---|---|---|---|---|---|---|---|---|
| | | | | Primary | Base | | Monochromatic | Y185 toning |
| | | | Specific | particle | adsorption | | evaluation: film | evaluation: |
| | | | conductivity | diameter | amount | Al content | thickness = 1.8 μm | (0.230, 0.670) |
| | Time | pH of | of pigment | of pigment | of pigment | of pigment | Contrast | Brightness |
| | (h) | pigment | (μS/cm) | (nm) | (mol/kg) | (mass ppm) | Std. ratio | Std. ratio |
| Example 13 | 8 | 7.5 | 64 | 24 | 0.140 | <1000 | 149% | 103.0% |
| Comparative | 8 | 3.4 | 167 | 31 | 0.118 | <1000 | Std. | Std. |
| Example 3 | | | | | | | | |

1. A method for producing a halogenated zinc phthalocyanine pigment, comprising:
a step of obtaining a halogenated zinc phthalocyanine crude pigment by deposition by extracting a halogenated zinc phthalocyanine, which is synthesized by using a compound that generates an acid by reacting with water, in a basic aqueous solution; and
a step of forming the halogenated zinc phthalocyanine crude pigment into a pigment.

2. The method according to claim 1, wherein a concentration of a basic compound contained in the basic aqueous solution is 1 mass % or more.

3. The method according to claim 1, wherein the basic aqueous solution contains a hydroxide of an alkali metal or alkaline earth metal.

4. The method according to claim 1, wherein the basic aqueous solution has a temperature of 5 to 90° C.

5. The method according to claim 1, wherein the halogenated zinc phthalocyanine crude pigment has a pH of 5.0 or more.

6. The method according to claim 1, wherein the halogenated zinc phthalocyanine crude pigment has an Al content of 3000 mass ppm or less.

7. A halogenated zinc phthalocyanine pigment having a base adsorption amount of 0.13 mol/kg or more and an Al content of 3000 mass ppm or less.
